(19) United States
(12) Patent Application Publication
(10) Pub. No.: US 2006/0275770 A1
(43) Pub. Date: Dec. 7, 2006

(54) Heart failure gene determination and therapeutic screening

(76) Inventor: Daniel Bednarik, Highland, MD (US)

Correspondence Address: FOLEY AND LARDNER LLP;SUITE 500, 3000 K STREET NW, WASHINGTON, DC, 20007, US

(21) Appl. No.: 10/547,931

(22) PCT Filed: Nov. 26, 2003
(86) PCT No.: PCT/US03/37927

§ 371 Date: Aug. 4, 2006

Related U.S. Application Data
(60) Provisional application No. 60/429,379, filed on Nov. 27, 2002.
(60) Provisional application No. 60/437,102, filed on Dec. 31, 2002.
(60) Provisional application No. 60/437,051, filed on Dec. 31, 2002.

(51) Int. Cl. C12Q 1/68; G01N 33/53; G01N 33/00

(52) U.S. Cl. 435/6; 435/7.1; 436/86

(57) ABSTRACT

Genes implicated in the progression of heart failure and in the endogenous myocardial recovery and repair mechanism provide the basis for screening potential therapeutic compounds for use as pharmaceutical preparations for the treatment of congestive heart failure. Target genes or target gene products whose expression level or biological activity are altered by a potential therapeutic compound can be validated functionally, by determining the physiological effect(s) precipitated by compound-induced changes in gene expression or biological activity of gene products. In particular, changes in the expression of the target gene(s) or in the biological activity of target gene product(s) can be correlated with indices of cardiac function, and a potential therapeutic compound that causes a therapeutically significant change such expression or activity warrants further clinical investigation as a drug candidate.

### CROSS-REFERENCE TO RELATED PATENT APPLICATIONS

This application claims the benefit of U.S. provisional application Nos. 60/429,379 filed Nov. 27, 2002, 60/437,102 filed Dec. 31, 2002, and 60/437,051 filed Dec. 31, 2002, which are incorporated by reference herein.

### FIELD OF THE INVENTION

The present invention relates to treatment of congestive heart failure. More specifically, the present invention relates to the identification of genes and gene products involved in the development and progression of heart failure as well as genes and gene products involved in the endogenous myocardial recovery and repair mechanism. The invention also relates to methods of identifying potential therapeutic compounds that alter the expression of such genes or the biological activity of the gene products.

### BACKGROUND OF THE INVENTION

Congestive heart failure affects an estimated 11 million Americans and causes more deaths, disabilities, and results in higher societal economic cost than any other disease in the developed world. Congestive heart failure is the most prevalent chronic life-threatening illness in the United States. Despite incremental advances in drug therapy, the prognosis for patients with advanced heart disease remains poor.

Congestive heart failure is a condition where the heart is not pumping effectively enough to meet the body's needs for oxygenated blood accompanied by a buildup of fluid in the lungs. Congestive heart failure can be of either ischemic or idiopathic etiology. The most common cause of congestive heart failure is ischemia due to arterial restriction resulting from atherosclerosis. Ischemia is a condition of oxygen insufficiency in a tissue where there is an imbalance between the supply and demand for oxygen in the tissue and perfusion is inadequate. Atherosclerosis reduces myocardial reperfusion by reducing the lumen of the coronary arteries and thus reducing blood flow. Other causes of reduced blood flow include arterial thrombi and spasm, as well as other less common events. Myocardial ischemia also may result from congenital abnormalities and may arise in situations where myocardial oxygen demand is increased markedly. Congestive heart failure is referred to as “idiopathic” when the etiology is unknown.

Irrespective of the etiology of heart failure, the diseased heart undergoes a process called “adaptive remodeling,” which includes morphological changes such as increased cell volume of cardiomyocytes (cellular hypertrophy), increased abundance of sarcomeres, and an overall increase in heart mass (hypertrophic cardiomyopathy). See, e.g., Houser et al., Trends Cardiovasc Med. 10:101 (2000). This phenotypic adaptation is paralleled by physiologically abnormal calcium homeostasis that compromises contractility by impairing the repolarization process in the myocardium. See Houser et al., supra; Bailey et al., Amer. J. Physiol. 265:H2009 (1993); Chen et al., Circ. Res. 91:517 (2002); Dipla et al., Circ. Res. 84:435 (1999); Gaughan et al., Amer. J. Physiol. 277:H714 (1999); Houser et al., J. Mol. Cell Cardiol., 32:1595 (2000); Nuss et al., Amer. J. Physiol. 263:H1161 (1992); Piacentino et al., J. Physiol. 523:533 (2000); Piacentino et al., Circ. Res. 90:435 (2002). It is at this stage of heart disease that adaptive remodeling becomes “maladaptive” by impeding normal cardiac function.

Patients afflicted with heart failure undergo a reversal of the heart disease phenotype after receiving circulatory support with left ventricular assist devices (LVADs). Chen et al., supra. LVAD support results in (1) improved cardiomyocyte contractility, faster relaxation, and improved calcium handling, (2) regression of pathologic cellular hypertrophy and associated expression of fetal genes and (3) recovery of action potential prolongation and patterns associated with sudden death. Thus, LVAD support induces functional and morphological recovery at the cellular level. These findings demonstrate that cells from even the most diseased human hearts retain a substantial degree of plasticity and capacity for recovery. The resultant improvement in the capacity of the heart to function in a physiologically normal manner is referred to as “reverse remodeling.”

The physiological changes that occur with the onset and progression of heart failure as well as the recovery of function via reverse remodeling are reflected as the genetic level. However, efforts to correlate these physiological changes with changes in gene expression have yielded little in the way of general insights. This may be due, in large part, to statistical difficulties due to small sample sizes in conjunction with limited clinical and/or physiological data.

For example, Tan et al., supra, compared microarray data from eight samples from human hearts diagnosed with end-stage dilated cardiomyopathy (DCM) with samples from eight non-failing human hearts. This study identified alterations in gene expression, but the limited number of samples and inherent biological variability between different hearts, plus the limited number of human genes on the microarray chip sets employed, rendered the results statistically suspect. Similarly, Barrans et al., Amer. J. Pathol. 6: 2035 (2002), examined global gene expression profiles in seven DCM hearts and five non-failing hearts. Here the researchers acknowledged the limitation of their study stating that “a larger n from our population would enhance the validity of our conclusions. Certainly, there exist no homogeneous heart failure genotype, especially among only seven DCM patients.” Id. at 2041.

Other studies of gene expression in heart failure have employed even smaller sample sizes. Hwang et al., Physiol. Genomics 10:31 (2002), compared gene expression in samples from three idiopathic DCM hearts and two hypertophic cardiomyopathy (HCM) hearts with three non-failing hearts. This study identified differences in gene expression between the failing versus non-failing hearts and also distinguished between differential expression in the DCM hearts as compared to the HCM hearts. Once again, the researchers admit that the veracity of the results may be hampered by the limited samples sizes.

Studies conducted to gain insight into changes in gene expression that correlate with LVAD-induced physiological changes with suffer from the statistical limitations as studies of heart failure discussed above. While the literature documents changes associated with reverse remodeling in myocardial gene expression and post-translational modification, it remains unclear whether particular changes at the molecular level are pivotal or epiphenomenal.

For example, Heerdt et al., Circulation 102: 2713 (2000), attempted to link LVAD-induced recovery of contractile strength with up-regulation of genes encoding proteins involved in regulating Ca²⁺ cycling, namely, sarcoplasmic endoreticular Ca²⁺-ATPase subtype 2a (SERCA2a) protein, sarcoplasmic reticular ryanodine-sensitive Ca²⁺ release channel (RyR) protein, and the sarcolemmal Na⁺—Ca²⁺ exchanger. Heerdt et al. found that, although all three genes were up-regulated after LVAD support, the level of protein increased for SERCA2a only. Even in a directed study of only a few genes, therefore, it was not possible to associate changes in gene expression with physiological changes related to LVAD-induced recovery.

There have been preliminary reports on genome-wide transcriptional profiling, to investigate changes in gene expression that occur with LVAD support. Tan et al., Circulation 102 (18 Suppl'mt): II.266 (2000); Rodrigue-Way et al., loc. cit.; Young et al., J. Am. College Cardiology 39 (Suppl'mt A): 199A (2002). These studies identified global alterations in gene expression but did not causally relate genetic changes as such to reverse remodeling or to the heart failure pathology that it affects.

Due to a lack of statistically rigorous gene expression profiling, the biological and clinical relevance of the observed changes in transcript levels during the onset, progression and reversal heart failure remains unclear.

### SUMMARY OF THE INVENTION

Among the large number of genes the expression of which may change during development and progression of heart disease and during the reversal of the disease phenotype, only a fraction, heretofore uncharacterized, is likely to comprise targets or to encode targets for therapeutic or diagnostic purposes. Accordingly, there is a need to illuminate the multigenic basis for various aspects of heart failure, including onset, progression, and LVAD-mediated reverse remodeling, in order to link alterations in gene expression or the biological activity of gene products with physiological phenomena in this context and to identify compounds that, by virtue of their impact on gene expression or alteration in a biological activity of a gene product, are potential therapeutic agents for treating heart failure.

To address these and other needs, the present invention provides methods of identifying genes and gene products involved in the development and progression of heart failure and as well as genes and gene products involved in the endogenous myocardial recovery and repair mechanism. The invention also provides methods of screening potential therapeutic compounds for cardiac therapeutic compositions and methods of using such therapeutic compounds.

Similarly, the present invention also provides a screening method to identify pharmaceutical compositions that alter the expression of genes or modulate the biological activity of gene products involved in the onset and progression of heart failure and genes involved in the endogenous myocardial recovery mechanism.

The invention further provides compounds that can affect the expression of genes involved in the development and progression of heart failure and genes involved in the endogenous myocardial recovery mechanism.

One embodiment of the invention is a method of screening potential therapeutic compounds for cardiac therapeutic preparations, comprising contacting a sample comprising a cell or tissue with a potential therapeutic compound, detecting a level of expression of a gene that codes for a product encoded by a nucleic acid selected from the group consisting of SEQ ID NOs: 1-98 and conservative variants thereof, and comparing the level of the expression of the gene to the level of expression of the gene in the absence of the compound. A potential therapeutic compound is identified as suitable for use as a cardiac therapeutic preparation if the potential therapeutic compound affects the level of expression of the gene.

Another embodiment of the invention is a method of screening potential therapeutic compounds for cardiac therapeutic preparations, comprising providing a sample comprised of (i) a cell or tissue comprising a gene product that is encoded by a nucleic acid selected from the group consisting of SEQ ID NOs: 1-98 and conservative variants thereof or (ii) said gene product in isolated form, contacting said sample with a potential therapeutic compound, measuring a level of biological activity of the gene product in the presence of the potential therapeutic compound and comparing the level of biological activity of the gene product to the biological activity of the gene product in the absence of the potential therapeutic compound. A potential therapeutic compound is identified as suitable for use as a cardiac therapeutic preparation if the biological activity of the gene product is modulated by the presence of the potential therapeutic compound.

A further embodiment of the invention is a pharmaceutical composition comprising a compound that affects the expression of at least one gene that codes for a product encoded by a nucleic acid selected from the aforementioned group of sequences and conservative variants thereof.

Another embodiment of the invention is a pharmaceutical composition comprising a compound that affects the biological activity of a gene product encoded by a gene that codes for a product encoded by a nucleic acid selected from the aforementioned group of sequences and conservative variants thereof.

A further embodiment of the invention is a method of treating heart failure in a subject in need thereof comprising administering to the subject a pharmaceutical composition comprising a compound that affects the expression of at least one gene that codes for a product encoded by a nucleic acid or the biological activity of such a product selected from the aforementioned group of sequences and conservative variants.

Other features, objects, and advantages of the present invention are apparent in the detailed description that follows. It should be understood, however, that the detailed description, while indicating preferred embodiments of the invention, are given by way of illustration only, not limitation. Various changes and modifications within the spirit and scope of the invention will become apparent to those skilled in the art from the detailed description.

### DETAILED DESCRIPTION OF THE PREFERRED EMBODIMENTS

The present invention is directed to an approach for identifying therapeutic compositions that modulate gene expression or the biological activity of a gene product associated with the development and progression of heart failure. The invention is also directed to a method of identifying therapeutic compositions that modulate gene expression or the biological activity of a gene product associated involved in endogenous myocardial recovery system.

Developing therapeutic pharmaceutical compositions in this regard requires an understanding of the changes in the expression of genes or the activity of gene products that drive the progression of and reversal of the heart failure phenotype.

An effective screening methodology for pinpointing compounds to use in developing such therapeutic compositions must be informed by correlations drawn between clinical/physiological variables, the expression patterns of myocardial genes, and the biological activity of myocardial gene products. That is, such correlations facilitate the identification of genes and gene products, the expression of and biological activity of which can be modulated by therapeutic compounds, in order to modulate the progression of heart failure or the recovery of heart function.

The success of gene expression analyses is dependent in part on the amount and quality of the data linking up-regulation or down-regulation of genes to physiological changes. GeneExpress® GX Explorer™ Training Manual, ver. 1.4, GeneLogic®, Inc., parts 1-9, App. A-G; Baxevanis and Francis-Ouellette, supra. Illustrative of the problems encountered in this regard, Razeghi et al., Cardiology 97: 203, 208 (2002), recognized that their small patient sample size (14 paired samples of diseased cardiac tissue with and without support mediated by a left ventricular assist device) probably was insufficient to accommodate the heterogeneity among subjects and prevented identification of changes in gene expression that are linked with clinical improvement. Furthermore, initial microarray studies of heart failure have illustrated that there is no homogeneous heart failure phenotype. Barrans et al., supra. Thus, meaningful correlations can only be drawn from statistically relevant sample sizes.

The present inventors have established a high-quality database of target genes in this regard, by accessing a large number of cardiac tissue samples from patients with heart disease, both before and after LVAD treatment. This database is not limited to left ventricle tissue samples, but includes all manner of cardiac tissue—left ventricle, right ventricle, left atrium, and right atrium. The database comprises 98 target genes, a gene product of which is listed in the accompanying TABLE. The target genes are those having expression levels that are altered in myocardium demonstrating heart failure and which also change during LVAD-mediated reverse remodeling. The number of tissue samples employed in compiling the database provides a foundation of statistical significance for analyzing the effect of potential therapeutic compounds on myocardial gene expression.

The TABLE associates (A) a sequence identification number (SEQ ID NO) for each of the genes with (B) a GeneBank accession number and the common nomeclature (gene identification name, name of encoded product, symbol, gene aliases) in common usage with respect to each gene. A compilation of database information appears in an APPENDIX hereto, providing publicly available data about the target genes, such as nucleotide sequence and encoded polypeptide sequence.

Knowledge of the relationship between discovered target genes to cellular pathways facilitates formulation of therapeutic approaches. The assessment of global alterations in mRNA levels in diseased tissue and the changes that occur during LVAD-mediated reversal of the disease phenotype provides insight into disease and recovery mechanisms and allows identification of novel candidates for therapeutic intervention with drugs. In particular, transcriptional profiling of cardiac tissue from patients with heart disease, compared to tissue from healthy (non-failing) heart, provides information regarding the genes whose expression levels change (upward or downward) during the development and progression of heart failure. Similarly, expression profiling of diseased cardiac tissue before and after treatment with LVAD identifies genes involved in the endogenous myocardial recovery system.

Identification of a compound that alters the expression level of a target gene or the biological activity of a target gene product can segue to functional validation of that gene or gene product, based on a determination of the physiological effect(s) precipitated by compound-induced changes in gene expression or activity of the gene product. For example, changes in the expression of the genes listed in the TABLE can be correlated with indices of cardiac function, such as cell volume, cell lengthening and shortening, Ca²⁺ transients, contractility, amount of sarcomeres, and changes in ion current. Houser et al., Trends Cardiovasc. Med. 10:101-7 (2000). A change in gene expression that correlates with recovery of normal cardiac function is termed “therapeutically significant” in the present description.

Similarly, the present invention contemplates identification of compounds that alter a biological activity of a gene product by, for example, affecting post-translational modification of the resulting protein, by inhibiting the enzyme activity of a target protein, by acting as a ligand to a target receptor protein, or by altering the affinity of a target receptor protein for its natural ligand. A change in biological activity can also be correlated with indicia of cardiac function. As discussed above with respect to gene expression, a change in a biological activity of a target gene product that correlates with improvement of cardiac function is also termed “therapeutically significant.”

A compound capable of causing such a therapeutically significant change in gene expression or in the activity of a target gene product is a potential pharmaceutical compound worthy of further clinical investigation, pursuant to the present invention.

Unless indicated otherwise, all technical and scientific terms are used in a manner that conforms to common technical usage. Generally, the nomenclature of this description and the described laboratory procedures, in cell culture, molecular genetics, and nucleic acid chemistry and hybridization, respectively, are well known and commonly employed in the art. Standard techniques are used for recombinant nucleic acid methods, oligonucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. Absent an indication to the contrary, the techniques and procedures in question are performed according to conventional methodology disclosed, for example, in Sambrook et al., MOLECULAR CLONING A LABORATORY MANUAL, 2d ed. (Cold Spring Harbor Laboratory Press, 1989), and CURRENT PROTOCOLS IN MOLECULAR BIOLOGY, John Wiley & Sons, 1989).

“Sequence identify” has an art-recognized meaning and can be calculated using published techniques. See COMPUTATIONAL MOLECULAR BIOLOGY, Lesk, ed. (Oxford University Press, 1988), BIOCOMPUTING: INFORMATICS AND GENOME PROJECTS, Smith, ed. (Academic Press, 1993), COMPUTER ANALYSIS OF SEQUENCE DATA, PART I, Griffin & Griffin, eds., (Humana Press, 1994), SEQUENCE ANALYSIS IN MOLECULAR BIOLOGY, Von Heinje ed., Academic Press (1987), SEQUENCE ANALYSIS PRIMER, Gribskov & Devereux, eds. (Macmillan Stockton Press, 1991), and Carillo & Lipton, SIAM J. Applied Math. 48:1073 (1988). Methods commonly employed to determine identity or similarity between two sequences include but are not limited to those disclosed in GUIDE TO HUGE COMPUTERS, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTP, BLASTN, FASTA (Atschul et al., J. Mol. Biol. 215:403 (1990)), and FASTDB (Brutlag et al., Comp. App. Biosci. 6: 237 (1990)).

I. Screening of Potential Therapeutic Compounds

A “target gene” is one that is implicated in the development and progression or LVAD-mediated reversal of of heart failure. A “target gene product” is a gene product encoded by a target gene and includes RNA and protein. Reference levels of target gene products or target gene product activity can be obtained by determining the levels of gene products and the activity of gene products in subjects displaying normal (non-failing) physiological function of the heart.

As noted, the TABLE identifies target genes, corresponding to SEQ ID NOs: 1-98, the expression levels of which change as a function of the development of or reversal of heart disease. Within the “target gene” category, the present invention also includes conservative variants of the genes of SEQ ID NOs: 1-98. A “conservative variant” is a nucleotide that hybridizes under stringent conditions (see below) to a oligonucleotide probe that, under comparable conditions, also binds to a gene of SEQ ID NOs: 1-98, respectively (“the parent gene”). A conservative variant nucleotide preferably exhibits at least about a 75 percent sequence identity with its parent gene.

Throughout this description, reference is made to the genes encoding the nucleic acid sequences of the TABLE, SEQ ID NOs: 1-98 or target genes and target gene products. “Target gene” refers to a gene, the expression of which results in a nucleic acid whose sequence is any of SEQ ID NO: 1-98. “Target gene product” refers to a gene product encoded by a gene that encodes the nucleic acid of any of SEQ ID NOs: 1-98. Unless otherwise indicated, an embodiment of the invention is a preferred subset of this group, namely, SEQ ID NOs: 6-7, 18-19, 21-23, 25-26, 29-32, 36, 42-45, 48-51, 55-57, 62, 64-83, 88-90, 92, 94-95, and 97-98 and their respective conservative variants.

In another embodiment, a subset of target genes includes SEQ ID NOs: 1-5, 7-9, 25-28, 41, 45, 48-50, 53-58, 68, 85-86, 91, and 93-94 and their respective conservative variants. This subset of genes includes genes which encode adenylate cyclase (SEQ ID NOs: 1 and 2), Ca²⁺ transporting and Na⁺/K⁺ transporting ATPases (SEQ ID NOs: 3-5), cell cycle, transcriptional and developmental regulating proteins (SEQ ID NOs: 7, 27, 28, 57, and 94), voltage-dependent calcium channel proteins (SEQ ID NOs: 8 and 9), proteins involved in cell signaling (SEQ ID NOs: 25-26), proteins implicated cardiac hypertrophy (SEQ ID NOs: 41, 45, 57), mineral corticoid receptors (SEQ ID NOs: 48-50), G-proteins (SEQ ID NO: 68), G-protein coupled receptors (SEQ ID NOs: 53-56), phospholamban (SEQ ID NO: 58), ryanodine receptors (SEQ ID NOs: 85 and 86), secreted frizzled protein (SEQ ID NO: 91), and a sodium/calcium exchanger (SEQ ID NO: 93). Modulation of expression of one or more of these genes or biological activity of a product encoded by one or more of these genes can be used to prevent, treat or improve function in a subject afflicted with heart failure.

For example, adenylate cyclases, which catalyze the formation of cAMP from ATP, are useful targets for therapeutic intervention in heart failure because cAMP regulates the activation of protein kinases that phosphorylate and activate or deactivate the L-Type calcium channel and/or phospholamban. Phospholamban, in turn, is capable of inhibiting cardiac muscle sarcoplasmic reticulum Ca²⁺-ATPase. The function of sarcoplasmic reticulum Ca²⁺-ATPase affects cardiac muscle relaxation rates and thus, phospholamban is a key regulator of cardiac function. The direct regulation of phospholamban, thus, is a also a pivotal therapeutic regulatory site for the control of calcium homeostasis. Similarly, Ca²⁺ transporting and Na⁺/K⁺ transporting ATPases, as well as voltage-dependent calcium channels and sodium/calcium exchangers, function to regulate calcium homeostasis. Dysregulation of calcium homeostasis in cardiomyocytes is central to the contractile dysfunction in heart failure. Therefore, compounds that normalize this process are potential therapeutic modalities.

Genes and the products of genes involved in the cell cycle, cell development, cell signaling are also useful targets for the treatment of heart failure because these target genes are pivotal in cardiomyocyte cell growth and development and, in part, drive cardiomyocyte hypertrophy and myogenesis. Mineral corticoid receptors also thought to be involved in transcriptional regulation and, thus, constitute suitable targets for the treatment of heart failure

A further embodiment of the invention provides a subset of target genes which includes SEQ ID NOs: 7, 25, 26, 45, 4849, 50, 55-57, 68, 82, and 94 and their respective conserved variants.

Another embodiment, a subset of target genes includes SEQ ID NOs: 7-9,12, 25-26, 41, 68, and 82 and their respective conserved variants.

In an additional embodiment of the invention provides a subset of target genes which includes SEQ ID NOs: 7, 25-26, 68, and 82 conservative variants thereof.

In another embodiment, a subset of target genes includes SEQ ID NOs: 26, 68, and 82 and their respective conserved variants. For example, the present invention identifies changes in the expression levels of the following genes after support with LVAD: the dual specificity phosphatase 5 (DUSP5; SEQ ID NO: 26), regulator of G-protein signaling 4 (RGS4; SEQ ID NO: 82), and a dexamethasone-induced RAS (SEQ ID NO: 68). The expression of these target genes modulate or are modulated by extracellular signal regulated kinase-1 (ERK), a mitogen-activated protein kinase (MAP kinase). DUSP5 deactivates ERK, RGS4 is a negative regulator of ERK. Furthermore, the expression of ATP2A2 (SERCA2; SEQ ID NO: 5) is regulated by ERK. In formulating a therapeutic approach, for example, in addition to targeting the RGS proteins directly in a therapeutic method, the activity of the RGS proteins can be modulated by targeting proteins that interact with the RGS proteins, such as PIP3, a natural inhibitor of RGS proteins, or antagonists of calmodulin, an activator of RGS.

RASD1 (SEQ ID NO: 68), which is stimulated by dexamethasone, encodes a G-protein that is highly expressed in human non-failing myocardium. Progression of heart failure results in reduction and/or inactivation of RASD1 expression. This early signaling event is linked to downstream processes which are determinant of cardiomyocyte hypertrophy and atrophy. Thus, increased expression of RASD1 provides a means of preventing or treating heart failure.

The initial screening of potential therapeutic compounds involves contacting a cell or tissue with a compound and then observing the effect on expression of target gene or genes or the effect on biological activity of a target gene product. “Gene expression” connotes the process of transcription of a DNA sequence into an RNA sequence, followed by translation of the RNA into a protein, which may or may not undergo post-translational processing. Thus, the effect of a potential therapeutic compound on gene expression can be observed by detecting, quantitatively or qualitatively, changes in the level of an RNA or a protein (“gene products”). “Biological activity” includes, but is not limited to, the activity of a protein gene product, including enzyme activity and receptor binding activity.

Screening of potential therapeutic compounds can be performed in vitro or in vivo. In vitro screening can be performed by contacting cells or tissues, from a subject or in a culture, with a compound and observing gene expression or activity of a gene product in the presence and absence of the compound. Typically, the compound is added to the cell medium or tissue culture medium. The amount of compound added and the length of exposure will depend on the particular characteristics of the compound. Compound concentrations can range from about 1 pg/mL to 1000 mg/mL. The compound to be tested can be supplied as an aqueous solution or can be solubilized in a solvent, such as DMSO, or a combination of solvents.

Screening of potential therapeutic compounds can be performed in vivo by obtaining cells or tissue samples from a subject before and after administration of a potential therapeutic compound and comparing gene expression or activity of gene products in the sample before and after administration of the potential therapeutic compound. Preferred subjects for in vivo studies are mammals. Preferred mammals include human, mouse, rat, guinea pig, or dog. The amount of potential therapeutic compound and method of administration will depend on the characteristics of the compound and can be readily determined by experimentation that is routine to the field.

A. Cell, Tissue, Nucleic Acid, and Protein Samples

Cells and tissues are preferentially isolated from cardiac tissue biopsy samples or from peripheral blood. In a preferred embodiment, the invention is practiced using cardiomyocytes, isolated trabeculae, or papillary muscle.

Gene products, including nucleic acid and amino acid gene products, can be isloated from cell fragments or lysates by any method known in the art. It is desirable to isolate gene products from cellular components capable of interfering with an analytical technique, such as a hybridization assay, enzyme assay, or ligand binding assay.

Nucleic acid samples used in practicing the invention can be prepared by any available method or process. Conventional techniques for isolating nucleic acids are detailed, for example, in Tijssen, LABORATORY TECHNIQUES IN BIOCHEMISTRY AND MOLECULAR BIOLOGY: HYBRIDIZATION WITH NUCLEIC ACID PROBES, chapter 3 (Elsevier Press, 1993), Berger and Kimmel, Methods Enzymol. 152:1 (1987), and GIBCO BRL & LIFE TECHNOLOGIES TRIZOL RNA ISOLATION PROTOCOL, Form No. 3786 (2000).

A suitable nucleic acid sample can contain nucleic acid derived from the transcript of a target gene, i.e., a nucleic acid for which synthesis of mRNA transcript or a subsequence thereof ultimately has served as a template. A cDNA reverse-transcribed from a transcript, an RNA transcribed from that cDNA, a DNA amplified from the cDNA, an RNA transcribed from the amplified DNA, etc., are exemplary of a nucleic acid derived from the transcript, and detection of such derived products is indicative of the presence and/or abundance of the original transcript in a sample. Thus, suitable samples include but are not limited to transcripts of the gene or genes, cDNA reverse-transcribed from the transcript, cRNA transcribed from the cDNA, DNA amplified from the genes, and RNA transcribed from amplified DNA. The category of “transcripts” includes but is not limited to pre-mRNA nascent transcripts, transcript processing intermediates, and mature mRNAs and degradation products thereof.

It is not necessary to monitor all types of transcripts to practice this invention. For example, one can choose to implement an embodiment of the invention to measure mature mRNA levels only.

In a preferred embodiment, a chromosomal DNA or cDNA library (e.g., fluorescently labeled cDNA synthesized from total cell mRNA) is prepared for use in hybridization methods according to recognized methods in the art. See Sambrook et al., supra.

It is desirable to inhibit or destroy RNase that often is present in homogenates or lysates, before used in hybridization techniques. Methods of inhibiting or destroying nucleases are well known. In one embodiment, cells or tissues are homogenized in the presence of chaotropic agents to inhibit nuclease. In another embodiment, RNase is inhibited or destroyed by heat treatment, followed by proteinase treatment.

Protein samples can be isolated by any means known in the art. Protein samples used in the invention can be crude cell lysates or crude tissue homogenates. Alternatively, proteins can be purified. Various methods of protein purification well known in the art may be found in Marshak et al., STRATEGIES FOR PROTEIN PURIFICATION AND CHARACTERIZATION: A LABORATORY COURSE MANUAL (Cold Spring Harbor Laboratory Press 1996).

B. Detecting Levels of Gene Expression

Methods of the invention involve detecting the level of gene expression. Any method for observing gene expression can be used, without limitation. These methods include traditional nucleotide hybridization techniques, polymerase chain reaction (PCR) based methods, and protein determination. Methods used in the present invention include solid support-based and solution-based assay formats.

Absolute measurements of the expression levels need not be made, although they can be made. Comparisons of differences in expression levels between samples is, however, preferred. Comparison of expression levels can be done visually or manually, or may be automated and done by a machine, using for example optical detection means. Subrahmanyam et al., Blood. 97: 2457 (2001); Prashar et al., Methods Enzymol. 303: 258 (1999). Hardware and software for analyzing differential expression of genes are available, and are preferentially used in practicing the present invention. GeneExpress® GX Explorer™ Training Manual, supra; Baxevanis & Francis-Ouellette, supra; GENE CHIP EXPRESSION MONITORING, Affymetrix GeneChip® Expression Analysis Technical Manual, 1999-2002, section 2, chapter 1-4.

An embodiment of the invention uses nucleic acid hybridization techniques to observes gene expression. These techniques include Northern blotting, Southern blotting, solution hybridization, and S1 nuclease protection assays.

Nucleic acid hybridization involves contacting a probe and target nucleic acid under conditions where the probe and its complementary target can form stable hybrid duplexes through complementary base pairing. For example, see PCT application WO 99/32660; Berger & Kimmel, Methods Enzymol. 152: 1 (1987). The nucleic acids that do not form hybrid duplexes are then washed away leaving the hybridized nucleic acids to be detected, typically through detection of an attached detectable label. In one embodiment, the target nucleic acids are detectably labeled polynucleotides representing the mRNA transcripts present in a cell (e.g., a cDNA library). Detectable labels are commonly radioactive or fluorescent labels, but any label capable of detection can be used. Labels can be incorporated by several approached described, for instance, in WO 99/32660, supra.

Duplexes of nucleic acids are destabilized by increasing the temperature or decreasing the salt concentration of the buffer containing the nucleic acids. Under low stringency conditions (e.g., low temperature and/or high salt) hybrid duplexes (e.g., DNA:DNA, RNA:RNA or RNA:DNA) will form even where the annealed sequences are not perfectly complementary. Thus, specificity of hybridization is reduced at lower stringency. Conversely, at higher stringency (e.g., higher temperature and/or lower salt and/or in the presence of destabilizing reagents) successful hybridization requires fewer mismatches.

Typically, stringent conditions for short probes (e.g., 10 to 50 nucleotides) will be those in which the salt concentration is at least about 0.01 to 1.0 M at pH 7.0 to 8.3 and the temperature is at least about 30° C. Stringent conditions may also be achieved with the addition of destabilizing agents such as formamide.

It may be desirable to perform hybridization at conditions of low stringency in, e.g., 6× SSPE-T (0.9 M NaCl, 60 mM NaH₂PO₄, pH 7.6, 6 mM EDTA, 0.005% Triton) at 37° C. to ensure hybridization. Subsequent washes can then be performed at higher stringency (e.g., 1× SSPE-T at 37° C.) to eliminate mismatched hybrid duplexes. Successive washes can be performed at increasingly higher stringency (e.g., down to as low as 0.25× SSPE-T at 37° C. to 50° C.) until a desired level of hybridization specificity is obtained. In a preferred embodiment, the wash is performed at the highest stringency that produces consistent results and that provides a signal intensity greater than approximately 10% of the background intensity.

In general, there is a compromise between stringency (hybridization specificity) and signal intensity. Thus, in a preferred embodiment, the hybridized array may be washed at successively higher stringency solutions and read between each wash. Analysis of the data sets produced in this manner will reveal a wash stringency above which the hybridization pattern is not appreciably altered and which provides adequate signal for the particular oligonucleotide probes of interest.

1. Probes

Probes useful in nucleic acid hybridization techniques are capable of binding to a target nucleic acid of complementary sequence through one or more types of chemical bonds, usually through complementary base pairing via hydrogen bond formation. A probe may include natural bases (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

Oligonucleotide probes can be prepared by any means known in the art. Probes useful in the present invention are capable of hybridizing to a nucleotide derived from the transcript of a target gene. Probes specific to the target genes of the invention can be generated using the nucleotide sequences disclosed in SEQ ID NOs: 1-98. The probes are preferably at least a 2, 10,12, 14, 16, 18, 20, 22, 24, or 25 nucleotide fragment of a corresponding contiguous sequence of SEQ ID NOs: 1-98, and can be less than 2, 1, 0.5, 0.1, or 0.05 kb in length.

Sequence-specific probe regions are defined within the coding and 3′ UTR for each gene to be detected. The probes complementary to the defined regions can be synthesized chemically, generated from longer nucleotides using restriction enzymes, or can be obtained using techniques such as polymerase chain reaction (PCR). The probes can be labeled, for example, with a radioactive, biotinylated, or fluorescent tag. PCR methods are well known and are described, for example, in Innis et al. eds., PCR PROTOCOLS: A GUIDE TO METHODS AND APPLICATIONS, Academic Press Inc. San Diego, Calif. (1990). Oligonucleotide probes generated by the above methods can be used in solution or solid support-based methods.

2. Oligonucleotide Array Methods

A preferred embodiment of the invention uses solid support-based oligonucleotide hybridization methods. Solid support-based methods suitable for practicing the present invention are widely known and are described, for example, in PCT application WO 95/11755; GENE CHIP EXPRESSION MONITORING, Affymetrix GeneChip® Expression Analysis Technical Manual, supra; Huber et al., Anal. Biochem. 299: 24 (2001); Meiyanto et al., Biotechniques. 31: 406 (2001); Relogio et al., Nucleic Acids Res. 30:e51 (2002). Any solid surface to which oligonucleotides can be bound, covalently or non-covalently, can be used. Such solid supports include filters, polyvinyl chloride dishes, silicon or glass based chips, etc.

A preferred embodiment uses oligonucleotide arrays which can be used to simultaneously examine a number of genes or gene products. Oligonucleotide arrays contain a particular oligonucleotide probe in a predetermined location on the array. Each predetermined location may contain more than one molecule of a probe, but each molecule within the predetermined location has an identical sequence. Such predetermined locations are termed features. There may be, for example, from 2, 10, 100, 1,000, 10,000, 100,000 or 400,000 of such features on a single solid support.

Oligonucleotide probe arrays for gene expression monitoring can be made and used according to conventional techniques described, for example, in Lockhart et al., Nat'l Biotech. 14: 1675 (1996), and McGall et al., Proc. Nat'l Acad. Sci. USA 93: 13555 (1996). Oligonucleotide arrays also are commercially available as prefabricated chips. For example, the Affymetrix GeneChip® Human Genome U133 Set is suitable for this method. A variety of oligonucleotide array designs is suitable for the practice of this invention.

A preferred embodiment of the invention employs an oligonucleotide array that includes test probes that specifically hybridize to nucleotides derived from the transcript of a target gene. Test probes can be oligonucleotides that range from about 5 to about 45 or 5 to about 500 nucleotides, more preferably from about 60 to about 100 nucleotides and most preferably from about 15 to about 40 nucleotides in length.

In a preferred embodiment, probes used in the microarray techniques are generated using PCR. PCR primers used in generating the probes are chosen, based on the sequences of SEQ ID NOs:1-98, to result in amplification of unique fragments (i.e., fragments that do not share more than 10 bases of contiguous identical sequence with any other fragment on the microarray). Computer programs are useful in the design of primers with the required specificity and optimal amplification properties. For example, see Oligo version 5.0 (National Biosciences).

In a particularly preferred embodiment, the oligonucleotide array will include one or more control probes. The control probes fall into two categories referred to herein as (1) normalization controls and (2) expression level controls.

Normalization controls are oligonucleotide or other nucleic acid probes that are complementary to labeled reference oligonucleotides or other nucleic acid sequences that are added to the nucleic acid sample to be screened The signals obtained from the normalization controls, after hybridization, provide a control for variations in hybridization conditions, label intensity, reading efficiency and other factors that may cause the signal of a perfect hybridization to vary between arrays. In a preferred embodiment, signals (e.g., fluorescence intensity or radioactivity) read from all other probes in the array are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes present in the array, but they also can be selected to cover a range of lengths. Further, the normalization control(s) can be selected to reflect the average base composition of the other probes in the array. In a preferred embodiment, only one or a few probes are used, and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

The terms “background” or “background signal intensity” refer to hybridization signals resulting from non-specific binding or other interactions between the labeled target nucleic acids and components of the oligonucleotide array. Background signals also can be produced by intrinsic fluorescence of the array components themselves.

A single background signal can be calculated for the entire array, or a different background signal can be calculated for each target nucleic acid. In a preferred embodiment, background is calculated as the average hybridization signal intensity for the lowest 5 to 10 percent of the probes in the array, or, where a different background signal is calculated for each target gene, for the lowest 5 to 10 percent of the probes for each gene. Where the probes to a particular gene hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background also can be calculated as the average signal intensity produced by regions of the array that lack any probes at all.

3. PCR-Based Methods

In another preferred embodiment PCR-based methods are used to detect gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, QUANTITATIVE PCR PROTOCOLS (Humana Press, 1999); Innis et al., supra.; Vandesompele et al., Genome Biol. 3: RESEARCH0034 (2002); Stein, Cell Mol. Life Sci. 59: 1235 (2002).

In a preferred embodiment, gene expression is observed in solution using Q-RTPCR. Q-RTPCR relies on detection of a fluorescent signal produced proportionally during amplification of a PCR product. See Innis et al., supra. Like the traditional PCR method, this technique employs PCR oligonucleotide primers, typically 15-30 bases long, that hybridize to opposite strands and regions flanking the DNA region of interest. Additionally, a probe (e.g., TaqMan®, Applied Biosystems) is designed to hybridize to the target sequence between the forward and reverse primers traditionally used in the PCR technique. The probe is labeled at the 5′ end with a reporter fluorophore, such as 6-carboxyfluorescein (6-FAM) and a quencher fluorophore like 6-carboxy-tetramethyl-rhodamine (TAMRA). As long as the probe is intact, fluorescent energy transfer occurs which results in the absorbance of the fluorescence emission of the reporter fluorophore by the quenching fluorophore. As Taq polymerase extends the primer, however, the intrinsic 5′ to 3′ nuclease activity of Taq degrades the probe, releasing the reporter fluorophore. The increase in the fluorescence signal detected during the amplification cycle is proportional to the amount of product generated in each cycle.

The forward and reverse amplification primers and internal hybridization probe must be designed to hybridize specifically and uniquely with one nucleotide derived from the transcript of a target gene. In a preferred embodiment, the selection criteria for primer and probe sequences incorporates constraints regarding nucleotide content and size to accommodate TaqMan® requirements.

The nucleic acid sequence of primer and probe oligonucleotides selected for each segment are queried in a BLAST search of GenBank to confirm that the selected primer and probe sequences are unique and complementary to the segment of each target gene.

In the present invention, preferentially each primer pair and probe will be employed to facilitate Q-RTPCR in duplicate, and more preferentially, in triplicate for each target gene. In a preferred embodiment multiple wells are combined with a robotic station to automate the process.

SYBR Green® can be used as a probe-less Q-RTPCR alternative to the Taqman®-type assay, discussed above. ABI PRISM® 7900 SEQUENCE DETECTION SYSTEM USER GUIDE APPLIED BIOSYSTEMS, chap. 1-8, App. A-F. (2002).

A device such as ABI 7900 Prism (Applied BioSystems, CA) measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (see U.S. Pat. No. 5,593,867).

4. Protein Detection Methods

Proteins can be observed by any means known in the art, including immunological methods, enzyme assays and protein array/proteomics techniques.

Measurement of the translational state may be performed according to several protein methods. For example, whole genome monitoring of protein—the “proteome”—can be carried out by constructing a microarray in which binding sites comprise immobilized, preferably monoclonal, antibodies specific to a plurality of protein species encoded by the genes of SEQ ID NOs:1-98. See Wildt et al., Nature Biotechnol. 18: 989 (2000). Methods for making polyclonal and monoclonal antibodies are well known, as described, for instance, in Harlow & Lane, ANTIBODIES: A LABORATORY MANUAL (Cold Spring Harbor Laboratory Press, 1988).

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al, GEL ELECTROPHORESIS OF PROTEINS: A PRACTICAL APPROACH (IRL Press, 1990). The resulting electropherograms can be analyzed by numerous techniques, including mass spectrometric techniques, western blotting and immunoblot analysis using polyclonal and monoclonal antibodies, and internal and N-terminal micro-sequencing.

C. Detecting Levels of Gene Product Activity

The effect of a potential therapeutic compounds on the biological activity of a gene product encoded by a target gene can also be examined. This biological activity includes, e.g., enzyme activity where the gene product is an enzyme and binding of a ligand to a receptor where the gene product is a receptor molecule.

1. Enzyme Assays

In enzyme-based screening assays, a sample containing an enzyme encoded by a target gene is contacted with a potential therapeutic compound. A sample can be an isolated gene product, a purified gene product, or a cell or tissue containing said gene product. Activity of the enzyme is measured in the presence of the potential therapeutic compound and compared to the activity of the enzyme in the absence of the potential therapeutic compound. An increase or decrease in the activity being measured is an indication that the potential therapeutic compound is suitable for use as a cardiac therapeutic compound. A potential therapeutic compound is considered to have an effect on enzyme activity if the activity being measured is increased or decreased preferably about 2-fold, more preferably at least about 5-fold, and most preferably at least about 10-fold or more, relative to the activity of the enzyme activity measured in the absence of the substance being tested.

Conditions and times sufficient for interaction of an enzyme with a potential therapeutic compound will vary with the enzyme, however, conditions generally suitable for interaction to occur is between about conditions generally suitable for interaction to occur is between 0° C. and about 40° C., preferably between 0° C. and about 37° C. The enzyme assay is preferably conducted in a buffered solution containing appropriate ions in an appropriate concentration range. These conditions will vary for a given enzyme; and will typically be within a pH range of between 5 and 9. Sufficient time for the binding and response will generally be between about 1 millisecond and about 72 hours after exposure.

For use in a screening assay, an enzyme can be present in a cell or tissue preparation and can be substantially assayed in said cell or tissue preparation, or first isolated from said cell or tissue preparation prior to being assayed. Preferably, an enzyme can be substantially purified. A “substantially isolated” or “purified” enzyme is one that is substantially free of the materials with which it is associated in nature, particularly of other proteinaceous material or substances which may inhibit an enzymatic activity related to reverse-remodeling. By substantially free is meant at least 50%, preferably at least 70%, more preferably at least 80%, and even more preferably at least 90% free of the materials with which it is associated in nature. A “substantially isolated” enzyme also refers to recombinant enzymes, which, by virtue of origin or manipulation: (1) are not associated with all or a portion of an enzyme with which it is associated in nature, (2) are linked to a polypeptide other than that to which it is linked in nature, or (3) does not occur in nature.

Enzymes for use in these assays can be obtained by any method known in the art, including, but not limited to, isolation from natural sources and production by recombinant techniques. An enzyme for use in the screening assays of the invention can be purified by any of the wide variety of known methods. See, e.g., Deutscher, Methods Enzymol. 182, chapter 1-61 (1990).

Enzyme activity can be determined by any known method including, but not limited to photometric, radiometric, HPLC and electrochemical techniques, which are described in, for example, ENZYME ASSAYS: A PRACTICAL APPROACH, eds. R. Eisenthal and M. J. Danson, Oxford University Press (1992).

2. Ligand Binding Assays

Ligand binding assays can be used to identify potential therapeutic compounds which affect binding of another compound such as a ligand to a receptor protein encoded by a target gene. Additionally, the potential therapeutic compound can be ligand itself. As used in the instant description “ligand” refers generally to all molecules capable of specifically recognizing or binding to a receptor molecule in vitro, on a target cell or in vivo. Specifically, examples of natural ligands include, but are not limited to, immunoglobulins or binding fragments thereof, lymphokines, cytokines, cell surface antigens such as CD22, CD4 and CD8, solubilized receptor proteins such as soluble CD4, hormones, growth factors such as epidermal growth factor (EGF), and the like which specifically bind desired target cells. Additionally, a potential therapeutic compound can also act as a ligand. Assays designed to measure the binding of a ligand to a receptor protein are well-established.

Ligand binding can be detected by any method known in the art, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, CURRENT PROTOCOLS IN MOLECULAR BIOLOGY, John Wiley & Sons, NY (1999). Ligands can be detectably labeled with a radiolabel, fluorescent label, a chemiluminescent label, or a bioluminescent label. Examples of suitable radioligands include ligands having one or more atoms enriched in a radioisotope, and ligands that are covalently coupled to a radioisotope label. Examples of radioisotopes that can be used to enrich ligands include ³H and ¹⁴C. Examples of radioisotopes that may be used to covalently label ligands include ¹²⁵I, ¹³¹I, ³²P, ³⁵S, ⁵¹Cr, ³⁶Cl, ⁵⁷Co, ⁵⁸Co, ⁵⁹Fe, and ⁵⁸Se. The ligands to be screened include, but are not limited to, compounds of extracellular, intracellular, biological or chemical origin.

For example, a binding assay can be conducted in which the binding of a natural ligand to a target protein gene product is detected in the presence of a potential therapeutic compound and compared to binding of the ligand in the absence of the potential therapeutic compound. A change in the extent of binding of the ligand in the presence of the potential therapeutic compound is indicative of interaction of the compound with the receptor and that compound is identified as worthy of further investigation. By the same methods potential therapeutic compounds can also be tested in vitro by determining the ability of a potential therapeutic compound to act as a ligand itself.

In one form of assay, a receptor encoded by a target protein incubated with labeled ligand, and a potential therapeutic compound is tested by measuring the ability of the compound to displace the labeled ligand bound to the receptor protein.

3. Signaling Assays

The binding of a natural ligand or a potential therapeutic compound capable of acting as a ligand to a receptor protein can result in signaling by a G protein-coupled receptor, and the activity of G proteins as well as other intracellular signaling molecules is stimulated. The induction of signaling function by a compound (e.g., a natural ligand or a potential therapeutic compound) can be monitored using any suitable method.

G protein activity, such as hydrolysis of GTP to GDP, or later signaling events triggered by receptor binding, such as induction of rapid and transient increase in the concentration of intracellular (cytosolic) free Ca²⁺ can be assayed by methods known in the art or other suitable methods. See e.g., Neote, et al., Cell 72: 415 (1993); Van Riper et al., J. Exp. Med. 177: 851 (1993); Dahinden, et al., J. Exp. Med. 179: 751 (1994). For example, the functional assay of Sledziewski et al., U.S. Pat. No. 5,284,746, using hybrid G protein coupled receptors, can be employed to monitor the ability a ligand or potential therapeutic compound to bind to a receptor protein and activate a G protein.

II. Functional Validation of Target Genes and Gene Products

The target gene and/or gene product can be validated functionally, to illuminate further its potential as a target for a cardiac therapeutic agent. Thus, “functional validation” denotes the process, according to the invention, of correlating a target gene to a therapeutically significant physiological response, by changing the expression of a target gene or activity of a target gene product that the compound was shown to affect. In this context, functional validation can be performed in vitro or in vivo, as explained below.

Techniques which can be employed in accordance with the present invention, to garner information about effects of gene expression on phenotypes associated with reverse remodeling include, but are not limited to: (i) over-expressing a gene product, (ii) disrupting a gene's transcript, such as by disrupting a gene's mRNA transcript; (iii) disrupting the function of a polypeptide encoded by a gene, or (iv) disrupting the gene itself. Over-expression of a gene product, the use of antisense RNAs, ribozymes, and the use of double-stranded RNA interference (dsRNAi) are valuable techniques for discovering the functional effects of a target gene and for generating gene knockouts.

Over-expression of a target gene often is accomplished by cloning the gene or cDNA into an expression vector and introducing the vector into recipient cells. Alternatively, over-expression can be accomplished by introducing exogenous promoters into cells to drive expression of genes residing in the genome. The effect of over-expression on cell function, biochemical and physiology properties can then be evaluated.

Antisense RNA, ribozyme, and dsRNAi technologies typically target RNA transcripts of genes, usually mRNA. Antisense RNA technology involves expressing in, or introducing into a cell, an RNA molecule (or RNA derivative) that is complementary to, or antisense to, sequences found in a particular mRNA into a cell. By associating with the mRNA, the antisense RNA can inhibit translation of the encoded gene product. Similarly, a ribozyme is an RNA that has both a catalytic domain and a sequence that is complementary to a particular mRNA. The ribozyme functions by associating with the mRNA (through the complementary domain of the ribozyme) and then cleaving (degrading) the message using the catalytic domain.

RNA interference (RNAi) involves a post-transcriptional gene silencing (PTGS) regulatory process, in which the steady-state level of a specific mRNA is reduced by sequence-specific degradation of the transcribed, usually fully processed mRNA without an alteration in the rate of de novo transcription of the target gene itself. The RNAi technique is discussed, for example, in Elibashir, et al., Methods Enzymol. 26: 199 (2002); McManus & Sharp, Nature Rev. Genetics 3: 737 (2002); PCT application WO 01/75164; Martinez et al., Cell 110: 563 (2002); Elbashir et al., supra; Lagos-Quintana et al., Curr. Biol. 12: 735 (2002); Tuschl et al., Nat Biotechnol. 20:446 (2002); Tuschl, Chembiochem. 2: 239 (2001); Harborth et al., J. Cell Sci. 114: 4557 (2001); et al., EMBO J. 20:6877 (2001); Lagos-Quintana et al., Science. 294: 8538 (2001); Hutvagner et al., loc cit, 834; Elbashir et al., Nature. 411:494 (2001).

The RNAi technique takes advantage of a regulatory mechanism, apparently at work in all cells, in which small interfering duplex RNA molecules (siRNAs), created by an endonuclease called a “dicer,” forms a complex with the target gene-encoded mRNA called the “RNA-induced silencing complex” or RISC. The targeted homologous mRNAs are subsequently degraded. The mediators of sequence-specific mRNA degradation are 21- to 23-nucleotide small interfering RNAs (siRNAs) generated by ribonuclease III cleavage from longer dsRNAs. Twenty-one-nucleotide siRNA duplexes trigger specific gene silencing in mammalian somatic cells without activation of the nonspecific interferon response.

In an embodiment of the invention, the RNAi technique is used to test whether a target gene or genes are involved in the development and progression of ischemic heart disease (see Example 2). This method links indicia of cardiac function, as discussed above, with the expression of a target gene.

Inhibition of mRNA and protein expression will be facilitated by RNAi methodology as described in INVITROGEN LIFE TECHNOLOGIES BLOCK-IT™ DICER RNAI KITS, Invitrogen Manual Catalog Nos. K3600-01 and K3650-01, Version A, Jul. 29, 2003 (25-0662), pp. 1-37 or in Tuschl et al., Chembiochem. 2: 239 (2002). In a preferred embodiment, an RNAi assay is performed in cardiomyocytes, isolated trabeculae or papillary muscle obtained from any mammal, but preferably from human, dog, rat, mouse, or guinea pig. The cells or tissues can be transfected with the target-specific siRNAs using any suitable method known in the art, such as lipofection or adenoviral infection. The targets are defined as any of the subset of SEQ ID NOs:1-98 whose expression levels are affected by a potential therapeutic compound. In a preferred embodiment, the RNAi effect on the physiological state of the cardiomyocytes will be assessed using Ca²⁺ transient assays.

III. Cardiac Therapeutic Compounds

The present invention also is directed to compounds capable of affecting the expression of target genes or the biological activity of target gene products. A compound identified by the methods described above as capable of modulating expression or activity of a target gene or gene product, respectively and validated as therapeutically functional, may be administered to a subject in a therapeutic application. In this regard, administering an amount of a compound of the instant invention that is effective in modulating the expression of a target gene or activity of the gene product represents a therapeutic application.

Therapeutic compounds identified with the current invention can be administered alone or in combination with other drug therapies including, but not limited to, β-adrenergic receptor antagonists, endothelial receptor antagonists, calcium channel antagonists, phosphodiesterase inhibitors, and/or angiotensin converting enzyme (ACE) inhibitors.

Pharmaceutical compositions identified with the current invention can be, but are not limited to, peptides, small molecules, large molecules, natural products, antibodies or antisense oligonucleotides.

Administration to the subject can be accomplished in any suitable manner known in the field. The pharmaceutical compositions of the present invention can be administered in the form of injectable compositions or formulated into well known compositions for any route of drug administration (e.g., oral, rectal, parenteral (intravenous, intramuscular, or subcutaneous), intracisternal, intravaginal, intraperitoneal, local (powders, ointments or drops), or as a buccal or nasal spray. A typical composition for such purpose comprises a pharmaceutically acceptable carriers, such as aqueous solutions, non-toxic excipients, including salts, preservatives, buffers and the like, as described, for instance, in REMINGTON'S PHARMACEUTICAL SCIENCES, 15th Ed. Easton: Mack Publishing Co. pp. 1405-1412 and 1461-1487 (1975), and THE NATIONAL FORMULARY XIV., 14th Ed. Washington: American Pharmaceutical Association (1975).

Examples of non-aqueous solvents in this context are propylene glycol, polyethylene glycol, vegetable oil and injectable organic esters such as ethyl oleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobials, anti-oxidants, chelating agents and inert gases. The pH and exact concentration of the various components of the pharmaceutical composition are adjusted in accordance with routine practice in this field. See GOODMAN AND GILMAN'S THE PHARMACOLOGICAL BASIS FOR THERAPEUTICS, 7th ed.

The following examples are given to illustrate the present invention. It should be understood, however, that the invention is not to be limited to the specific conditions or details described in these examples.

### EXAMPLE 1

### Transcriptional Validation

An extensive validation protocol to confirm a change in gene expression as the result of a potential therapeutic compound is employed. Expression profile validation is conducted in the liquid phase using real time, quatitative reverse-transcription polymerase chain reaction (Q-RTPCR). Although the validation quality of the Affymetrix U133 microarray data via GeneExpress® analysis demonstrates high reliability, many of the probe characteristics cannot take into account some important biological issues such as alternative splice variation and/or multiple polyadenylation sites. Q-RTPCR affords a semi-automated approach to assessing chemigenomic changes induced by treatment of in vitro functional validation model systems, thereby establishing a crucial method of identifying and mapping changes in biological pathways.

Sequence-specific probe regions are defined within the coding and 3′ UTR for each target gene. TaqMan® probes are designed using the sequence-specific probe regions. Probes are typically 60-100 nt long. The primer pair for each target gene is employed to facilitate Q-RTPCR in triplicate.

A robotic station is coupled to a ABI 7900 Prism PCR instrument from Applied Biosystems (Foster City, Calif.). The ABI 7900 Prism possesses a built-in thermal cycler with a 384-well sample format. The following fluorescent dyes can be used as reporter fluorophores: 6-FAM -(PE Biosystems) which has an excitation wavelength of 494 nm and an emission wavelength of 525 nm, TET (PE Biosystems) which has an excitation wavelength of 521 nm and an emission wavelength of 536 nm, HEX (PE Biosystems) which has an excitation wavelength of 535 nm and an emission wavelength of 556 nm, Cy5 (Amersham Pharmacia Biotech) which has an excitation wavelength of 643 nm and an emission wavelength of 667 nm, Cy3 (Amersham Pharmacia Biotech) which has an excitation wavelength of 535 nm and an emission wavelength of 552 nm and an emission wavelength of 570 nm.

### EXAMPLE 2

### Analysis of Data for Transcriptional Validation

In this example, the GeneExpress® Software System Fold Change Analysis tool is used to identify genes expressed at least 1.5-fold in the up and down directions in diseased myocardium compared to normal (non-failing) heart. For each gene fragment, the ratio of the geometric means of the expression intensities in the diseased myocardium compared to normal (non-failing) heart tissue is calculated and the fold change then calculated on a per fragment basis. Confidence limits are calculated using a two-sided Welch modified t-test on the difference of the means of the logs of the intensities.

GeneExpress® supports a “Gene Logic normalization” methodology. The algorithm for this normalization approach is based on the observation that the expression intensity values from a single-chip experiment have different distributions, depending on whether small or large expression values are considered. Small values, which are assumed to be mostly noise, are approximately normally distributed with mean zero, while larger values roughly obey a log-normal distribution. In other words, their logarithms are normally distributed with some nonzero mean.

Whereas Affymetrix normalization applies the same scale factor to all expression values in an experiment, Gene Logic normalization computes separate scale factors for non-expressors (small values) and expressors (large ones). The inputs to the algorithm are the Affymetrix-normalized average difference values, which are scaled already to set the trimmed mean equal to 100. The algorithm computes the standard deviation SDnoise of the negative values, which are assumed to come from non-expressors. It then multiplies all negative values, as well as all positive values less than 2.0*SDnoise, by a scale factor proportional to 1/SDnoise. Values greater than 2.0*SDnoise are assumed to come from expressors. For these values, the standard deviation SD log(signal) of the logarithms is calculated. The logarithms then are multiplied by a scale factor proportional to 1/SD log(signal) and exponentiated. The resulting values are multiplied by another scale factor, chosen so that there will be no discontinuity, in the normalized values from unscaled values, on either side of 2.0*SDnoise.

Detection p-Values

The absolute expression analysis in the Affymetrix MAS version 5.0 software introduces the concept of a detection p-value, in addition to the absolute call and the absolute expression intensity. For each probe set on the array, this analysis generates these three measures related to the determination of absolute expression. The detection p-values should not be confused with the p-values generated as part of the fold change and contrast analysis algorithms, which are based on multiple measurements across user-specified sample sets. These analysis p-values reflect the statistical power of multiple observations in the Gene Logic expression database to give significance estimates for differences in expression of particular transcripts across many conditions. The detection p-values rely on single measurements of individual transcripts taken in isolation, so they provide information only about that one particular sample, whereas the fold change and contrast analysis p-values give information about trends and patterns across many samples. The detection p-values measure the significance of the absolute call by performing a statistical test on the individual probe pair measurements. These p-values are used to determine the absolute call by comparing the computed p-value against a threshold. If the detection p-value is less than 0.04, then the gene is called present. If the detection p-value is between 0.04 and 0.06, the nth gene is called marginal, and if the detection p-value is greater than 0.06, the gene is called absent.

Normalization

Normalization aids in accurate comparison of expression data from different GeneChip experiments. The process of normalization reduces the effects of variability introduced into the system due to differences in sample preparation, hybridization conditions, staining or use of different lots of arrays. GeneExpress® 2000 supports five different methods of normalization: 1) Affymetrix normalization, MAS 4.0.2) Affymetrix normalization, MAS 5.0.3) Gene Logic normalization 4) Standard Curve or Spike-In normalization for MAS 4.0 and Standard Curve for MAS 5.0.

Affymetrix normalization is a global scaling method, where the overall intensity of the chip affects the scale factor. The top 2% and bottom 2% of all expression intensity values on the chip are discarded, and the remaining 96% of values are used to compute the “trimmed mean.” The scale factor (SF) is then calculated using this adjusted mean (SF=100/trimmed mean), and this single scale factor is applied to the expression values for every fragment on a given chip to produce normalized average difference values (Average difference).

Normalization uses as input the normalized average difference values that are generated by the Affymetrix method of normalization, but divides them into two distinct groups. The small expression intensities, including the negative values, are considered to be non-expressors, and their average differences are normally distributed with mean of zero. The larger intensity values reflect the expression levels of genes that are actually expressed in the sample. The normalized average differences of these expressors are log-normally distributed about some mean greater than zero. A standard deviation is calculated for the smallest expression values, such that the non-expressors are scaled to a nominal value of 20. The expressors are those fragments with intensity values greater than twice the standard deviation of the non-expressors, or greater than 40. The standard deviation is used to figure the scale factor. Finally, the normalized expression value from this method is calculated by multiplying the scale factor by the Average difference for non-expressors, or by the natural log of the Average difference for expressors.

Normalization using a standard curve, or spike-in normalization, is a method that relates the original expression intensity values from a chip to the actual concentration of mRNA for each gene expressed in a given sample. This is accomplished by spiking into the hybridization mix a number of bacterial gene fragments of known concentration. The concentration (pM) of the known fragments are then plotted against the unscaled average difference values, and a linear regression line is drawn. The slope of this line can then be used to convert Average difference values to concentration (pM). It is important to note that in order to use this method of normalization all chips within a sample set must have these bacterial genes spiked-in.

The new statistical algorithms, referred to as MAS 5.0, were designed by Affymetrix to reflect more accurately the distribution of data from microarray experiments. Expression values are calculated based on the hybridization intensities of each probe pair within a probe set representing a single transcript. These intensity measurements are expressed in both a quantitative and qualitative manner. For MAS 5.0, the quantitative measurement is given as SIGNAL, the relative abundance of a given transcript, as opposed to AVERAGE DIFFERENCE in the previous empirical algorithms (MAS 4.0 and earlier versions). The qualitative measure has been referred to as ABSOLUTE CALL, and is now described as DETECTION. Detection continues to be assessed as PRESENT, ABSENT or MARGINAL calls. Associated with this detection measurement is an indication of statistical significance, the DETECTION P-VALUE. These values reflect the confidence of a detection call, giving the user a clearer determination of the statistical significance of the detection call. MAS 5.0 algorithms also compute both upper and lower confidence intervals in relation to the detection p-values.

Explanation of Signal

Signal, the quantitative metric calculated for each probe pair, represents the level of expression of a given transcript. It is calculated using a standard statistical technique known as One Step Tukey's Biweight Estimate which returns a weighted mean that is relatively insensitive to outliers. In this calculation, the mismatch (MM) of a probe pair is used as an estimate of background hybridization or stray signal. This background measurement is made according to a number of strict rules that relate the hybridization intensities of MM to PM to generate the estimated background for each probe pair, and ensure that the signal values will be greater than zero, more accurately representing the expression level of the transcript. The actual signal for a probe pair is determined by using the MM as a specificity control and comparing its hybridization intensity to the hybridization intensity of the corresponding perfect match (PM), as seen in the formula:
SIGNAL=log PM−estimated stray signal

Each probe pair is then assigned a weight according to how distinct the intensity difference is between PM and MM for that probe pair. In the statistical algorithm, however, unlike the empirical algorithm, not all probe pairs have equal influence on the determination of signal. A probe pair carries more weight in the overall signal calculation when it is closer to the median signal value for the probe set. The signal value that a user sees for any given probe set is the mean of these weighted intensity values.

Detection and Detection p-Values

Detection is measured as PRESENT, ABSENT or MARGINAL. Specific probe pair intensities are used to generate these detection calls and their associated p-values. Each individual probe pair within a probe set influences the detection call for that transcript. This influence, or “vote,” is referred to as the discrimination score, R, and is a measure of the ability of each probe pair to detect its intended target. More specifically, the discrimination score assesses the difference in hybridization intensities between PM and MM, relative to the overall intensity of the probe pair:
R=(PM−MM)/(PM+MM)

The target specific intensity (R) is then compared to a pre-defined threshold, tau, to determine whether the probe pair will vote Present or Absent.
R>tau, probe pair votes PRESENT
R<tau, probe pair votes ABSENT

The sum of the individual probe pair votes determines the detection p-value, using a One-Sided Wilcoxon's Signed Rank Test which assigns a rank to each probe pair based on the distance R is from threshold, tau. This p-value associated with each detection call indicates whether a transcript is reliably detected, in which case it would be called Present, or not detected, in which case it would be called Absent. As in any calculation with an associated p-value, the lower the detection p-value, the more confidence the user has in the PRESENT call. Conversely, the higher p-values, generally associated with ABSENT calls, show a statistically significant confidence that the transcript indeed is not present for that sample.

### EXAMPLE 3

### Functional Validation—RNA Inhibition

In this example, the RNAi method described in Tuschl et al., supra, is used to inhibit expression of genes identified as changing during transcriptional validation of potential therapeutic compounds.

Cardiomyocytes or isolated trabeculae or isolated papillary muscle from human or rat left ventricle are transfected with the target-specific siRNAs via lipofection or adenoviral infection. In the case of lipofection, efficiencies are determined by introduction of a plasmid containing the cytomegalovirus (CMV) promoter driving transcription of green fluorescent protein (GFP). Fluorescent imaging is employed to quantify the number of myocytes expressing GFP. Quantitation of GFP per ng total protein is ascertained by preparing a cell-free extract of transfected myocytes and subsequent serial dilution. Extracts are analyzed by quantifying fluorescence emission levels after excitation by 395 nm light, and normalized against a GFP standard.

Functional analyses of siRNA-transfected cardiomyocytes are approached by determining the inhibition of mRNA and/or protein synthesis and by measuring the RNAi effect on the physiological state of the cardiomyocytes via Ca²⁺ transients assay. Cardiomyocytes routinely harvested from adult and/or neonatal rat hearts are cultured in 96-well format. Cells transfected with target-specific siRNAs in triplicate are extracted to isolate total RNA and protein. Levels of mRNA are subsequently determined via Q-RTPCR analysis. Protein determinations are performed by two-dimensional gel electrophoresis, followed by western blot analysis using target protein-specific mono- or polyclonal antibodies.

This approach identifies targets that modulate calcium homeostasis and combines data reduction from three key biological levels, namely, transcriptional/post-transcriptional, post-translational and physiological processes integral to cardiomyocyte function. Additionally, acquisition of these data is facilitated in parallel through processing of each experiment.

### EXAMPLE 4

### Functional Validation—Assessment of Physiological Function Targetability for Target Genes in the Therapy of Human Heart Failure

This example employs a rabbit model of heart failure for validation of target genes in vitro and in vivo. Heart failure is mimicked in rabbits as previously described in Shannon et al., Circ. Res. 93:592 (2003). siRNA molecules directed to RNA encoded by SEQ ID NOs: 1-98 is synthesized either by obtaining the sequence of the known rabbit homologues from the public domain (GenBank), or by generating the equivalent gene sequence from rabbit myocardial RNA.

In the preliminary validation, short-term assessment of the effect(s) of in vitro knock-out on contractility and Ca²⁺ transients, myocytes isolated from nonfailing or failing left ventricles are subjected to RNAi gene transfer using the newly introduced pLenti6/BLOCK-iT-DEST lentiviral vector system. See INVITROGEN LIFE TECHNOLOGIES BLOCK-IT™ DICER RNAI KITS, Invitrogen Manual Catalog Nos. K3600-01 and K3650-01, Version A, Jul. 29, 2003 (25-0662), pp. 1-37.

Infection of myocytes with siRNA constructs is performed as described in Invitrogen Manual Catalog, Id. Transient analyses are performed using the IonOptix Sarclens system as described previously. Ren and Wold, Biol. Proceed. Online. 3:43-53 (2001).

To assess the effect(s) of RNAi-mediated gene knock-out in vivo, adenovirus constructs containing the siRNA of interest are prepared and gene transfer into the left ventricle of rabbit hearts is performed as described in Tevaearai et al., Circulation 106:124 (2002). Physiological characterization of cardiac output data is acquired in vivo using a Millar catheter and cardiomyocyte hypertrophy assessed via echocardiogram. See Thakral et al., J. Appl. Physiol. 89:1159 (2000), Moran et al., Herz. 28:52 (2003).

TABLE 1
Heart Failure Gene List

| | GenBank | | | |
|---|---|---|---|---|
| SEQ ID | Accession | | | |
| NO | Number | Gene ID | Gene Name | Gene Aliases |
| 1 | NM_001114 | ADCY7 | adenylate cyclase 7 | KIAA0037 |
| 2 | NM_001116 | ADCY9 | adenylate cyclase 9 | |
| 3 | NM_000702 | ATP1A2 | ATPase, Na+/K+ | |
| | | | transporting, alpha 2 | |
| | | | (+) polypeptide | |
| 4 | NM_000703 | ATP1A3 | ATPase, Na+/K+ | |
| | | | transporting, alpha 3 | |
| | | | polypeptide | |
| 5 | NM_004320 | ATP2A1 | ATPase, Ca++ | ATP2A, |
| | | | transporting, cardiac | SERCA1 |
| | | | muscle, fast twitch 1 | |
| 6 | U87408 | B1 | PTH-responsive | |
| | | | osteosarcoma B1 | |
| | | | protein | |
| 7 | NM_006763 | BTG2 | BTG family, member 2 | PC3, TIS2I |
| 8 | NM_023035 | CACNA1A | calcium channel, | APCA, |
| | | | voltage-dependent, | CACNL1A4, |
| | | | P/Q type, alpha 1A | EA2, FHM, |
| | | | subunit | HPCA, MHP, |
| | | | | MHPI, SCA6 |
| 9 | NM_000718 | CACNA1B | calcium channel, | CACNL1A5, |
| | | | voltage-dependent, L | CACNN |
| | | | type, alpha 1B | |
| | | | subunit | |
| 10 | NM_000719 | CACNA1C | calcium channel, | CACNL1A1, |
| | | | voltage-dependent, L | CCHL1A1 |
| | | | type, alpha 1C | |
| | | | subunit | |
| 11 | NM_000720 | CACNA1D | calcium channel, | CACNL1A2, |
| | | | voltage-dependent, L | CCHLIA2 |
| | | | type, alpha 1D | |
| | | | subunit | |
| 12 | NM_000723 | CACNB1 | calcium channel, | CACNLBI, |
| | | | voltage-dependent, | CCHLBI |
| | | | beta 1 subunit | |
| 13 | NM_000724 | CACNB2 | calcium channel, | CACNLB2, |
| | | | voltage-dependent, | MYSB |
| | | | beta 2 subunit | |
| 14 | U07139 | CACNB3 | calcium channel, | CACNLB3 |
| | | | voltage-dependent, | |
| | | | beta 3 subunit | |
| 15 | NM_000726 | CACNB4 | calcium channel, | |
| | | | voltage-dependent, | |
| | | | beta 4 subunit | |
| 16 | NM_006549 | CAMKK2 | calcium/calmodulin- | CAMKK, |
| | | | dependent protein | CAMKKB, |
| | | | kinase kinase 2, beta | KIAA0787 |
| 17 | NM_001231 | CASQ1 | calsequestrin 1 (fast- | CASQ |
| | | | twitch, skeletal | |
| | | | muscle) | |
| 18 | NM_005093 | CBFA2T2 | core-binding factor, | EHT, MTGRI |
| | | | runt domain, alpha | |
| | | | subunit 2; | |
| | | | translocated to, 2 | |
| 19 | NM_001295 | CCR1 | chemokine (C-C | CKR-1, |
| | | | motif) receptor 1 | CMKBRI, |
| | | | | HM145, |
| | | | | MIP1aR |
| 20 | NM_001825 | CKMT2 | creatine kinase, | SMTCK |
| | | | mitochondrial 2 | |
| | | | (sarcomeric) | |
| 21 | NM_004362 | CLGN | calmegin | |
| 22 | U52696 | CREBL1, | cAMP responsive | CREB-RP, |
| | | TNXB | element binding | G13, HXBL, |
| | | | protein-like 1, | HXBL; XB, |
| | | | tenascin XB | TENX, TNX, |
| | | | | TNXB1, |
| | | | | TNXB2, |
| | | | | TNXBS, |
| | | | | TNXBS; XB, |
| | | | | XB, XBS |
| 23 | NM_001881 | CREM | cAMP responsive | |
| | | | element modulator | |
| 24 | NM_005213 | CSTA | cystatin A (stefin A) | STFI, STFA |
| 25 | NM_004417 | DUSP1 | dual specificity | CL100, HVHI, |
| | | | phosphatase 1 | MKP-1, |
| | | | | PTPN10 |
| 26 | U16996 | DUSP5 | dual specificity | HVH3 |
| | | | phosphatase 5 | |
| 27 | NM_001964 | EGR1 | early growth | KROX-24, |
| | | | response 1 | NGFI-A, TIS8, |
| | | | | ZIF-268 |
| 28 | U91903 | FRZB | frizzled-related | FRP-3, FRZB- |
| | | | protein | PEN, FRZBI, |
| | | | | SFRP3 |
| 29 | NM_003608 | GPR65 | G protein-coupled | HTDAG8, |
| | | | receptor 65 | TDAG8 |
| 30 | NM_022049 | GPR88 | G-protein coupled | |
| | | | receptor 88 | |
| 31 | NM_014888 | GS3786 | predicted osteoblast | |
| | | | protein | |
| 32 | NM_005734 | HIPK3 | homeodomain | DYRK6, PKY, |
| | | | interacting protein | YAK1 |
| | | | kinase 3 | |
| 33 | NM_002233 | KCNA4 | potassium voltage- | HK1, HPCN2, |
| | | | gated channel, | KCNA8, |
| | | | shaker-related | KV1.4 |
| | | | subfamily, member 4 | |
| 34 | U33428 | KCNAB1 | potassium voltage- | AKR6A3 |
| | | | gated channel, | HKVB3, |
| | | | shaker-related | HKVBETA3, |
| | | | subfamily, beta | KCNA1B |
| | | | member 1 | KVB1.3 |
| 35 | NM_012281 | KCND2 | potassium voltage- | KIAA1044, |
| | | | gated channel, Shal- | KV4.2, RK5 |
| | | | related subfamily, | |
| | | | member 2 | |
| 36 | NM_014592 | KCNIP1 | Kv channel | KCHIPI |
| | | | interacting protein 1 | |
| 37 | NM_014591 | KCNIP2 | Kv channel | KCHIP2 |
| | | | interacting protein 2 | |
| 38 | NM_002245 | KCNK1 | potassium channel, | DPK, HOHO, |
| | | | subfamily K, member | TWIK-1, |
| | | | 1 (TWIKI-1) | TWIK1 |
| 39 | NM_016611 | KCNK4 | potassium channel, | DKFZP566E1 |
| | | | subfamily K, member 4 | 64, TRAAK |
| 40 | U89364 | KCNQ1 | potassium voltage- | KCNA8, |
| | | | gated channel, KQT- | KCNA9, |
| | | | like subfamily, | KVLQT1 |
| | | | member 1 | LQT, LQT1 |
| 41 | AB059408 | LAGY | homeodomain only | Cameo, HOP, |
| | | | protein | SMAP31, |
| | | | | Toto |
| 42 | NM_006762 | LAPTM5 | Lysosomal-associated | |
| | | | multispanning | |
| | | | membrane protein-5 | |
| 43 | NM_015978 | LOC51086 | putative protein- | |
| | | | tyrosine kinase | |
| 44 | NM_016568 | LOC51289 | G-protein coupled SALPR | |
| | | | receptor SALPR; | |
| | | | somatostatin and | |
| | | | angiotensin-like | |
| | | | peptide receptor | |
| 45 | NM_016298 | LOC51725 | muscle disease- | |
| | | | related protein | |
| 46 | NM_004635 | MAPKAPK3 | mitogen-activated | 3PK, |
| | | | protein kinase- | MAPKAP3 |
| | | | activated protein | |
| | | | kinase 3 | |
| 47 | NM_017572 | MKNK2 | MAP kinase- | GPRK7, |
| | | | interacting | MNK2 |
| | | | serine/threonine | |
| | | | kinase 2 | |
| 48 | NM_000901 | NR3C2 | nuclear receptor | MCR, MLR, |
| | | | subfamily 3, group | MR |
| | | | C, member 2 | |
| 49 | NM_002135 | NR4A1 | nuclear receptor | GERPI, HMR, |
| | | | subfamily 4, group A, | NIG, NAK-1, |
| | | | member 1 | NGFIB, NP 10, |
| | | | | NUR77, TR3 |
| 50 | S77154 | NR4A2 | nuclear receptor | HZF-3, NOT, |
| | | | subfamily 4, group A, | NURR1, |
| | | | member 2 | RNR1, |
| | | | | TINOR, |
| | | | | TINUR |
| 51 | NM_003489 | NRIP1 | nuclear receptor | RIP14O |
| | | | interacting protein 1 | |
| 52 | NM_002563 | P2RY1 | purinergic receptor | P2Y1 |
| | | | P2Y, G-protein | |
| | | | coupled, 1 | |
| 53 | NM_002566 | P2RY1 | purinergic receptor | P2Y11 |
| | | | P2Y, G-protein | |
| | | | coupled, 11 | |
| 54 | NM_002564 | P2RY2 | purinergic receptor | HP2U, P2U, |
| | | | P2Y, G-protein | P2Y2 |
| | | | coupled, 2 | |
| 55 | NM_002565 | P2RY4 | pyrimidinergic | NRU, P2P, |
| | | | receptor P2Y, G- | P2Y4 |
| | | | protein coupled, 4 | |
| 56 | NM_004154 | P2RY6 | pyrimidinergic | P2Y6 |
| | | | receptor P2Y, G- | |
| | | | protein coupled, 6 | |
| 57 | NM_002616 | PER1 | period homolog 1 | PER, RIGUI |
| | | | (Drosophila) | |
| 58 | NM_002667 | PLN | phospholamban | PLB |
| 59 | NM_002481 | PPP1R12B | protein phosphatase | MYPT2 |
| | | | 1, regulatory | |
| | | | (inhibitor) subunit 12B | |
| 60 | NM_006741 | PPP1R1A | protein phosphatase | |
| | | | 1, regulatory | |
| | | | (inhibitor) subunit 1A | |
| 61 | NM_002711 | PPP1R3A | protein phosphatase | PPPIR3 |
| | | | 1, regulatory | |
| | | | (inhibitor) subunit 3A | |
| | | | (glycogen and | |
| | | | sarcoplasmic | |
| | | | reticulum binding | |
| | | | subunit, skeletal | |
| | | | muscle) | |
| 62 | NM_016203 | PRKAG2 | protein kinase, AMP- | AAKG, |
| | | | activated, gamma 2 | AAKG2, |
| | | | non-catalytic subunit | H91620P, |
| | | | | H91620p, |
| | | | | WPWS |
| 63 | NM_006587 | PRSC | corin | CRN |
| 64 | NM_007079 | PTP4A3 | protein tyrosine | PRL-3, PRL- |
| | | | phosphatase type | R, PRL3 |
| | | | IVA, member 3 | |
| 65 | NM_002829 | PTPN3 | protein tyrosine | PTPH1 |
| | | | phosphatase, non- | |
| | | | receptor type 3 | |
| 66 | NM_002831 | PTPN6 | protein tyrosine | HCP, HCPH, |
| | | | phosphatase, non- | HPTP1C, |
| | | | receptor type 6 | PTP-1C, SH- |
| | | | | PTP1, SHP-1, |
| | | | | SHP-1L |
| 67 | NM_002886 | RAP2B | RAP2B, member of | |
| | | | RAS oncogene | |
| | | | family | |
| 68 | AF069506 | RASD1 | RAS, | AGS1, |
| | | | dexamethasone- | DEXRAS1, |
| | | | induced 1 | MGC:26290 |
| 69 | NM_000321 | RB1 | retinoblastoma 1 | |
| | | | (including | |
| | | | osteosarcoma) | |
| 70 | NM_007369 | RE2 | G-protein coupled | |
| | | | receptor | |
| 71 | NM_014059 | RGC32 | RGC32 protein | |
| 72 | S59049 | RGS1 | regulator of G- | BL34, IER1, |
| | | | protein signalling 1 | IR20 |
| 73 | W19676 | RGS10 | regulator of G- | |
| | | | protein signalling 10 | |
| 74 | NM_003834 | RGS11 | regulator of a- | RS11 |
| | | | protein signalling 11 | |
| 75 | NM_006480 | RGS14 | regulator of G- | |
| | | | protein signalling 14 | |
| 76 | U94829 | RGS16 | regulator of G- | A28-RGS14, |
| | | | protein signalling 16 | RGS-r |
| 77 | NM_012419 | RGS17 | regulator of G- | RGS-17, |
| | | | protein signalling 17 | RGSZ2 |
| 78 | NM_005873 | RGS19 | regulator of G- | GAIP, |
| | | | protein signalling 19 | GNAI3IP- |
| | | | | PENDING, |
| | | | | RGSGAIP |
| 79 | NM_005716 | RGS19IP1 | regulator of G- | C19orf3, |
| | | | protein signalling 19 | GIPC, |
| | | | interacting protein I | GLUTICBP, |
| | | | | IIP-1, TIP-2 |
| 80 | NM_002923 | RGS2 | regulator of G- | GOS8 |
| | | | protein signalling 2, | |
| | | | 24kD | |
| 81 | NM_021106 | RGS3 | regulator of G- | C2PA, |
| | | | protein signalling 3 | FLJ20370, |
| | | | | PDZ-RGS3, |
| | | | | RGP3 |
| 82 | NM_005613 | RGS4 | regulator of G- | |
| | | | protein signalling 4 | |
| 83 | NM_002924 | RGS7 | regulator of G- | |
| | | | protein signalling 7 | |
| 84 | NM_003835 | RGS9 | regulator of G- | RGS9L |
| | | | protein signalling 9 | |
| 85 | NM_001035 | RYR2 | ryanodine receptor 2 | VTSIP |
| | | | (cardiac) | |
| 86 | NM_001036 | RYR3 | ryanodine receptor 3 | |
| 87 | NM_006271 | S100A1 | S100 calcium | S100, S100A |
| | | | binding protein A1 | |
| 88 | NM_002976 | SCN6A | sodium channel, | SCN7A |
| | | | voltage-gated, type | |
| | | | VI, alpha polypeptide | |
| 89 | NM_002977 | SCN9A | sodium channel, | NE-NA, NE- |
| | | | voltage-gated, type | NA;, PN1 |
| | | | IX, alpha polypeptide | |
| 90 | NM_001038 | SCNN1A | sodium channel, | ENaCa, |
| | | | nonvoltage-gated I | ENaCalpha, |
| | | | alpha | SCNN1 |
| 91 | NM_003012 | SFRP1 | secreted frizzled- | FRP, FRP-1, |
| | | | related protein 1 | FRP1, FrzA, |
| | | | | SARP2 |
| 92 | NM_016334 | SH120 | putative G-protein | GPR89- |
| | | | coupled receptor | PENDING |
| 93 | NM_021097 | SLC8A1 | solute carrier family | NCX1 |
| | | | 8 (sodium/calcium | |
| | | | exchanger), member 1 | |
| 94 | NM_003118 | SPARC | secreted protein, | ON |
| | | | acidic, cysteine-rich | |
| | | | (osteonectin) | |
| 95 | U17714 | ST13 | suppression of | FAM10A1, |
| | | | tumorigenicity 13 | HIP, HOP, |
| | | | (colon carcinoma) | HSPABP, |
| | | | (Hsp70 interacting | HSPABP1, |
| | | | protein) | P48, |
| | | | | PRO0786, |
| | | | | SNC6 |
| 96 | NM_003877 | STATI2 | STAT induced STAT | CIS2, SOCS- |
| | | | inhibitor-2 | 2, SSI-2, SSI2 |
| 97 | U12170 | TCF8 | transcription factor 8 | AREB6, BZP, |
| | | | (represses interleukin | NIL-2-A, ZEB |
| | | | 2 expression) | |
| 98 | NM_003407 | ZFP36 | zinc finger protein | GOS24, |
| | | | 36, C3H type, | NUP475, |
| | | | homolog (mouse) | TIS11, TTP |

1. A method of screening potential therapeutic compounds for cardiac therapeutic preparations, comprising:
contacting a sample comprising a cell or tissue with a potential therapeutic compound;
detecting a level of expression of a gene that codes for a product encoded by a nucleic acid selected from the group consisting of SEQ ID NOs: 1-98 and conservative variants thereof;
comparing the level of expression of the gene to the level of expression of the gene in the absence of the compound; and
identifying a potential therapeutic compound as suitable for use as a cardiac therapeutic preparation if the potential therapeutic compound affects the level of expression of the gene.

2.-74. (canceled)

75. The method of claim 1, wherein the product is selected from the group consisting of DNA, RNA, and protein.

76. The method of claim 75, wherein the DNA is cDNA.

77. The method of claim 75, wherein the RNA is mRNA.

78. The method of claim 75, wherein the product is a protein.

79. The method of claim 1, wherein the product is labeled with a detectable label.

80. The method of claim 1, wherein the step of detecting is selected from the group consisting of: a nucleic acid hybridization technique, a PCR technique, and a protein detection technique.

81. The method of claim 80, wherein the nucleic acid hybridization technique is performed on a solid support.

82. The method of claim 81, wherein the solid support comprises two or more oligonucleotide probes.

83. The method of claim 82, wherein the solid support further comprises two or more control probes, and wherein the control probes are selected from the group consisting of normalization control probes and expression level control probes.

84. The method of claim 82, where the two or more oligonucleotide probes are capable of hybridizing to one of the products encoded by a nucleic acid selected from the group consisting of SEQ ID NOs: 1-98 and conservative variants thereof.

85. The method of claim 82, wherein two or more of the oligonucleotide probes hybridize to a product encoded by a gene comprising a different one of the nucleic sequences selected from the group consisting of SEQ ID NOs: 1-98 and conservative variants thereof.

86. The method of claim 82, wherein the oligonucleotide probe is a fragment of a contiguous sequence of SEQ ID NOs: 1-98.

87. The method of claim 80, wherein the PCR technique is selected from the group consisting of: RT-PCR, real time RT-PCR, and Q-RT-PCR.

88. The method of claim 78, wherein the step of detecting is performed using a technique selected from the group consisting of: immunological methods, enzyme assays, and proteomics techniques.

89. A method of screening potential therapeutic compounds for cardiac therapeutic preparations, comprising:
providing a sample comprised of (i) a cell or tissue comprising a gene product that is encoded by a nucleic acid selected from the group consisting of SEQ ID NOs: 1-98 or (ii) said gene product in isolated form;
contacting said sample with a potential therapeutic compound;
measuring a level of biological activity of a gene product in the presence of the potential therapeutic compound;
comparing the level of biological activity of the gene product to the biological activity of the gene product in the absence of the potential therapeutic compound; and
identifying a potential therapeutic compound as suitable for use as a cardiac therapeutic preparation if the biological activity of the gene product is modulated by the presence of the potential therapeutic compound.

90. A pharmaceutical composition comprising a compound that affects the expression of or biological activity of at least one gene that codes for a product encoded by a nucleic acid selected from the group consisting of SEQ ID NOs: 1-98 and conservative variants thereof.

91. A method of treating heart failure in a subject in need thereof comprising administering to the subject a therapeutically effective amount of the pharmaceutical composition of claim 76.
